(19) United States
(12) Patent Application Publication
(10) Pub. No.: US 2004/0018575 A1
(43) Pub. Date: Jan. 29, 2004

(54) Sample preparation device and method

(76) Inventors: Craig Rappin, Long Grove, IL (US); Kiamars Hajizadeh, Lincolnshire, IL (US); Peter Lewis, Streamwood, IL (US); Kelly Mills, McHenry, IL (US)

Correspondence Address: ROGER H. STEIN, ESQ., WALLESTEIN & WAGNER, LTD., 53rd FLOOR, 311 SOUTH WACKER DRIVE, CHICAGO, IL, 60606-6630, US

(21) Appl. No.: 10/208,178

(22) Filed: Jul. 29, 2002

(51) Int. Cl. G01N 33/53; G01N 33/537; G01N 33/543

(52) U.S. Cl. 435/7.92

(57) ABSTRACT

A disposable device, a method of sample preparation, and a business method are provided for collecting and preparing a sample for subsequent direct analysis of a particular analyte. The device includes a sampling assembly for collecting a sample, a homogenizing body for comminuting the sample, and a container with a buffer. The homogenizing body has two sites for attachment—one site being attachable to the sampling assembly and the other, being attachable to the container. The device includes a first reagent and a second reagent to facilitate sample preparation, which may respectively be proteinase-K and proteinase-K inhibitor for preparing a sample for analysis of pathogenic prion protein. One embodiment includes a delivery apparatus for dispensing the second reagent into the treated homogenate. The delivery apparatus has a dropper top dispensing component with a pore at a top end, an elongated dispensing member attached inside the dispensing component and terminating in a tip outside the dispensing component, and proteinase-K inhibitor disposed on the tip. In another embodiment, the device comprises a housing defining a recess therein and having at least one opening for collecting a sample, and a sample-reaction zone separated from the recess by a sample-comminution zone. Also provided is method for collecting, comminuting, and optionally treating the homogenized sample to prepare it for direct analysis. Another aspect of the invention is a business method for preparing biological tissue from animals for prion analysis.

### TECHNICAL FIELD

This invention relates to devices and methods for preparing samples for analysis for a particular analyte. In particular, the invention relates to devices and methods for collecting and homogenizing a sample in a suitable medium and optionally treating the sample so it may be directly analyzed by a method of choice. The invention also relates to devices and methods for collecting and preparing food and biological samples by homogenization and proteinase-K treatment to remove interfering constituents so the treated sample may be directly analyzed for the pathogenic form of prion protein.

### BACKGROUND OF INVENTION

Before virtually any analytical procedure may be utilized, it requires that a representative test sample be collected from the material to be analyzed and that the sample be prepared in a manner appropriate for the analysis. The steps used to prepare the sample vary, depending upon the particular analyte. Generally, however, after a sample is obtained for analysis, one of the first steps involves reducing the sample matrix into smaller-sized units—whether that be particles, strands, or bits and pieces of the sample. At the same time, the sample is generally liquefied or diluted with a buffer so it may be introduced into the analytical procedure. Additional sample preparation may include extracting the desired analyte into the buffer.

A number of sample preparation methods are at the disposal of researchers. Some devices and methods are labor intensive and require extensive steps before yielding even a small aliquot of an “analysis-ready” sample. For example, separation techniques such as centrifugation may be burdensome, as they take time to fill the centrifuge tubes and load and unload the centrifuge.

Efforts have been made to simplify the extraction of analytes. For example, in U.S. Pat. No. 6,090,572, Croby discloses a device for filtering a biological fluid and extracting an analyte from the fluid. The device has a pliant body with a sealable open top and a gradient filter assembly. A biological fluid is introduced into the device through the open top, which is then sealed. By squeezing the device, the user creates a positive pressure that causes the biological fluid to flow through the filter so particulates—such as Chlamydia and/or Neisseria gonorrhea—may be captured on the filter. The top end may then be opened and a reagent, such as a protease extraction reagent, added. The top is re-sealed, and the user squeezes the pliant body again so the reagent flows through the filter assembly and extracts the desired analyte from the microorganisms held on the filter. The clarified liquid is then expressed through the opposite end of the device. Although this device works for biological liquids, it lacks utility for materials such as certain foods and biological tissues and organs and requires the user to add the requisite reagent(s) separately.

One example of an analyte acquiring greater attention today is pathogenic prion protein, which is implicated in a variety of transmissible neurodegenerative disorders that afflict both humans and animals. Prion diseases result from infection by prions—microorganisms that join bacteria, viruses, and viroids as pathogens. Examples of prion diseases afflicting animals include scrapie in sheep and goats, and bovine spongiform encephalopathy in cattle. Humans are susceptible to four prion diseases including kuru, Creutzfeldt-Jakob disease, Gerstmann-Strassler-Scheinker disease, and fatal familial insomnia.

The protein in prions occurs in both a normal form and a pathogenic form. The pathogenic form is the one associated with prion diseases.

When samples are prepared for analysis of pathogenic prion protein, the normal prion protein must be eliminated because its presence interferes with the analysis. Other factors making pathogenic prion protein difficult to detect include its poor solubility in many biological buffers and the tenacious resistance of its aggregates to dissolution. As a result, analytical procedures for detecting prion protein are oftentimes time-intensive and complex. For example, hydrophilic-interaction chromatography has been used to purify the abnormal prion protein, followed by capillary electrophoresis immunoassay for detection. See, Schmerr and Alpert, U.S. Pat. No. 6,150,172, citing Electrophoresis 19:409 (1998).

U.S. Pat. No. 6,180,417, issued to Hajizadeh and Wijesuriya, discloses an immunochromatographic assay for analysis of prion proteins. U.S. Pat. No. 4,703,017 issued to Campbell et al. and U.S. Pat. No. 5,591,645 issued to Rosenstein use visible particles in immunochromatography test strips. Neither the test strips nor the assays of these patents, however, provide for the preparation of samples containing pathogenic prion protein.

U.S. Pat. No. 6,150,172 issued to Schmerr and Alpert discloses a three-step method for extracting abnormal prion protein from homogenized biological material and analyzing the extracted protein with a chromatographic immunoassay. The extraction method includes incubating an aqueous preparation of the biological sample with a pre-measured amount of proteinase-K to digest the normal prion protein, isolating the pathogenic prion protein by mixing the pre-treated sample with an extraction solvent, and recovering the isolated pathogenic prion protein in the extraction solvent. Col. 4, lines 21-26. The extraction solvent may then be applied directly to a support and assayed via immunochromatography. Although the referenced method isolates and detects abnormal prion protein, it involves multiple steps and requires as much as two hours for merely extracting the analyte. Col. 9, lines 27-28 (referring to an extraction time of from 1 to 2 hours).

Thus, there exists a need for devices and simplified methods for preparing samples for analysis and particularly for preparing biological samples and food samples for direct analysis of pathogenic prion protein or other analytes. The present invention addresses such need.

### SUMMARY OF THE INVENTION

The present invention provides devices and methods for rapidly preparing samples for direct analysis of a particular analyte of interest. To this end, in one aspect of the invention, a device is provided for preparing a sample for analysis of pathogenic prion protein. The device includes a housing having a recess and a sample-reaction zone adapted to contain a buffer, a comminuting device, and proteinase-K. Also included are at least one opening in the housing for collecting a sample in the recess, a plunger or other suitable means for discharging the sample from the recess into the reaction zone, and a dispensing pore in the housing for dispensing the enzyme-treated sample for direct analysis of pathogenic prion protein. The comminuting device is chosen from (a) a plurality of pores on a homogenizing face disposed within one of the recess and the reaction zone and (b) a plurality of discrete bodies of an inert composition—such as glass beads or rods—disposed in the reaction zone in a quantity and having a dimension sufficient to comminute the sample to a suitable size for analysis. The proteinase-K is in the form chosen from a capsule, a pellet, a gel, a powder, a lyophilizate, an encapsulate, and a liquid, as examples. In one embodiment, the enzyme is in the buffer as an encapsulate, coated with a constituent that requires heat to release the enzyme.

According to a second aspect of the invention, a device is provided for collecting and homogenizing a sample for direct analysis of a particular analyte. The device includes a container adapted to hold a buffer, a homogenizing body, and a sampling assembly. The homogenizing body has an open end, an opposed end having a homogenizing face with at least one pore, and a bore therebetween. The homogenizing body is attachable to the container to form a homogenizing composite such that the at least one pore extends into the container. The sampling assembly has a housing defining a recess therein, means for collecting a sample in the recess, and means for discharging the sample through the at least one pore of the homogenizing body, such as a plunger slidable within the recess of the sampling assembly. The sampling assembly is typically inserted into the open end of the homogenizing body, opposite the site that attaches to the container. In this respect, the recess in the sampling assembly communicates with the bore of the homogenizing body.

In another embodiment, the device includes a first reagent for release into the container, such as an enzyme, a chemical, a biological agent, an antibody, a marker, a polymer, and any other suitable compound. Preferably, the first reagent is proteinase-K.

In yet another embodiment, the device includes a delivery apparatus for dispensing a second reagent—preferably, proteinase-K inhibitor—into the container. The delivery apparatus includes a dropper top dispensing component, an elongated dispensing member having a tip and being attachable to the dispensing component, and a second reagent disposed on the tip. The dispensing component has an apparatus housing defining a recess, a top end with a pore therethrough, and an opposed open end that is attachable to the container. The elongated member is attachable inside the apparatus housing and extends away from the top end and with the tip extending beyond the open end of the dispensing component. When the dispensing component is attached to the container, the tip of the elongated member extends at least partially into the container.

In accordance with a third aspect of the invention, a sample preparation device is provided which includes a container, a homogenizing body having a homogenizing face with at least one pore therein, and a sampling assembly, each substantially as described above. The device also includes a first reagent releasable into the container, and means for attaching the sampling assembly to the homogenizing body and for attaching the homogenizing body to the container so the at least one pore extends into the container. The homogenizing body has from 1 to 20 pores which may have a cross-sectional shape chosen from round, elliptical, oblong, amorphous, conical, or with n sides, n being an integer of 3 or greater.

In accordance with a fourth aspect of the invention, a sample preparation device is provided which includes a sampling assembly and a homogenizing body, each attachable to the other to form a sampling homogenizer. The homogenizing body has an open end, an opposed end including at least one comminuting pore, and a bore therebetween. The homogenizing body is further adapted to attach to a container, such as a vial, a test tube, or a beaker at the end opposite the attached sampling assembly. The sampling assembly includes a housing having a discharge pore, at least one opening along the longitudinal axis of the housing for collecting a sample, and a sleeve for sealing the at least one opening. In one embodiment, the sampling homogenizer may be a single unitary piece. In another embodiment, the device is fabricated of a rigid material by injection molding.

In a fifth aspect of the invention, a device is provided for preparing a food or biological sample for direct analysis of pathogenic prion protein. The device includes proteinase-K in an amount sufficient to substantially remove nonpathogenic prion from the sample, a reaction vessel adapted to contain a buffer, a homogenizing body substantially as described above, and a sampling assembly substantially as described above with a plunger slidable in the recess for discharging the sample through the at least one pore in the homogenizing body. The sampling assembly is attachable to the homogenizing body so the at least one opening is in communication with the bore of the homogenizing body. The device also includes proteinase-K inhibitor, structures for attaching the homogenizing body to the reaction vessel, and structures for attaching the sampling assembly to the homogenizing body. In one embodiment, the device includes a delivery apparatus for dispensing the proteinase-K inhibitor into the container.

In yet another aspect of the invention, a method is provided for preparing a sample for direct analysis for a particular analyte. The method may be used, e.g., to prepare a sample of biological material or food for direct analysis of pathogenic prion protein. The method includes providing a sample preparation device substantially as described above, collecting a sample through at least one opening of the at least one opening in the sampling assembly, and comminuting the sample by extruding the sample through the at least one pore into the buffer to form a homogenate that is ready of analysis. In one embodiment, the method includes adding a first reagent to the homogenate in an amount sufficient to facilitate sample preparation and, optionally, heating the homogenate under conditions sufficient to further prepare the homogenate for analysis. In another embodiment, the method further includes adding a second reagent to the homogenate and optionally heating the homogenate to a temperature for a time sufficient to allow the second reagent to act upon the sample and form a treated, analysis-ready sample. In one embodiment, where the analyte of interest is pathogenic prion protein, the first reagent is proteinase-K and the second reagent is proteinase-K inhibitor.

In a further aspect, a method suitable for use by meat processors and others is provided for testing a biological sample, such as brain tissue from an animal, for prion disease. The method is used with a device substantially as discussed above. The method includes (a) providing a device labeled with an identification code of the animal whose tissue is being tested; (b) dissolving the proteinase-K in the buffer in a container attached to the homogenizing body; (c) collecting a sample with the sampling assembly; (d) securing the sampling assembly to the homogenizing body; (e) comminuting the sample by discharging the sample through the at least one pore into the buffer to form an enzyme-containing homogenate; (f) in a first heating step, heating the enzyme-containing homogenate in the assembled device at a temperature ranging from about 30 to about 40 degrees Centigrade for from about 30 seconds to about 30 minutes to remove interfering constituents and yield an enzyme-treated sample; (g) releasing the proteinase-K inhibitor into the enzyme-treated sample upon attaching the dispensing apparatus to the container in place of the homogenizing body and sampling assembly composite; (h) in an optional second heating step, heating the inhibitor-containing sample to a temperature of up to about 100 degrees Centigrade for from about 1 minute to about 30 minutes to yield an analysis-ready sample; and (i) dispensing the analysis-ready sample into a testing device utilizing immunochromatography. In one embodiment, the testing device is a lateral flow device.

Other aspects of the invention will become apparent when taken in conjunction with the following description and drawings.

### BRIEF DESCRIPTION OF THE DRAWINGS

To understand the present invention, it will now be described by way of example, with reference to the accompanying drawings in which:

FIG. 1 is a perspective view of a buffer-containing sample preparation device and vial cap made in accordance with the teachings of the present invention;

FIG. 2 is a cross-sectional view of the homogenizing body and the container of the device of FIG. 1, taken along lines 2-2 of FIG. 1;

FIG. 3 is a enlarged cross-sectional view of a portion of a homogenizing body, showing the homogenizing face with pores in a frustoconical configuration;

FIG. 4 is an enlarged perspective view of the homogenizing body showing the pores configured as a sieve;

FIG. 5 is a perspective view of a homogenizing composite fabricated as a single unit;

FIG. 6 is cross-sectional view of the sampling assembly 14, taken along lines 6-6 of FIG. 1;

FIG. 7 is a cross-sectional view of a composite sample comminuter, comprising a sampling assembly and a homogenizing body as a single unit;

FIG. 8 is a cross-sectional view of a delivery apparatus for releasing a second reagent into the container;

FIGS. 9a and 9b are cross-sectional views of two embodiments of a delivery apparatus wherein FIG. 9a shows a solid elongated dispensing member having a second reagent disposed on the surface, and FIG. 9b shows an elongated dispensing member having the second reagent within a bore running along at least a portion of the longitudinal axis of the dispensing member;

FIG. 10 is a cross-sectional view of a dispenser comprising the delivery apparatus of FIG. 8 and a container 12; and,

FIGS. 11a and 11b are cross-sectional views of two further embodiments of the device wherein comminution of the sample is provided by a homogenizing face as shown in FIG. 11a, and a plurality of discrete bodies of an inert composition as shown in FIG. 11b.

### DETAILED DESCRIPTION OF THE INVENTION

While this invention is susceptible of embodiments in many different forms, preferred embodiments of the invention are illustrated in the drawings and described in detail herein, with the understanding that the present disclosure is to be considered as an exemplification of the principles of the invention and is not intended to limit the broad aspect of the invention to the embodiments illustrated.

This invention is directed to devices and methods for preparing a sample for subsequent and direct analysis of an analyte of choice that requires extraction from a larger sample matrix, including, e.g., pathogenic prion protein, pathogens such as Escherichia coli and Salmonella species, metals, environmental toxins, agricultural additives such as pesticides and herbicides, genetic markers in grains, and food constituents. Throughout this application, the following terms have the meanings set forth below.

“Biological material” and “biological sample” refer to the fluid or tissue extracted from vertebrates, such as animals or humans, and may include brain tissue, blood, serum, plasma, nasal secretions, vaginal secretions, saliva, urine, bladder washings, feces, tonsils, spleen, colon washings, cerebral spinal fluid, or fluid from body systems such as the respiratory, circulatory, reproductive, and digestive, as examples.

“PrP^{C}” refers to the non-diseased form of prion protein, which is removed in-situ by proteinase-K (PK) enzyme from the samples.

“PrP^{SC}” refers to the pathogenic prion protein which is the analyte in methods for detecting a prion disease in a vertebrate.

A. The Devices

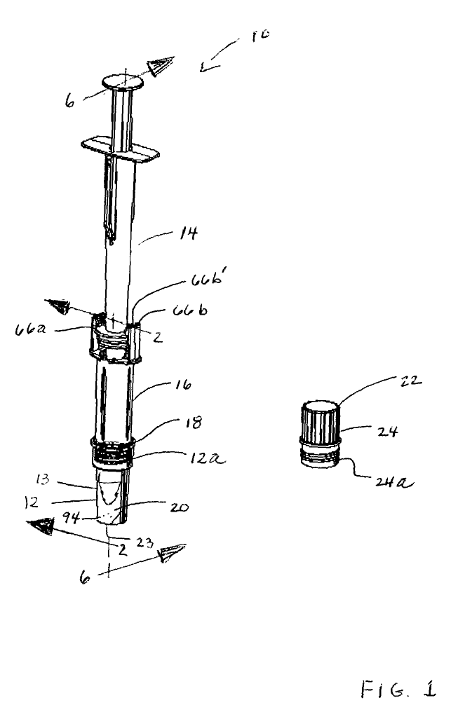

Shown in FIG. 1 is a sample preparation device 10 made in accordance with a first embodiment of the invention. The sample preparation device 10 is typically a disposable, easy-to-operate assembly used to collect and comminute a sample into smaller particle sizes or strands by extruding the sample as fine streams into a buffer where it may be treated with at least one reagent to prepare it for subsequent analysis. The sample preparation device 10 includes a container 12 adapted to contain a buffer 20, a sampling assembly 14 for collecting a sample, and a homogenizing body 16 having two attachable sites—one site being attachable to the sampling assembly 14 and the other site being attachable to the container 12 for comminuting the collected sample into the container 12.

Specifically, the container 12 is transparent so one can see through the side wall 13 whether it contains a liquid. In an alternative embodiment, container 12 is opaque. The container 12 has an open top end 18, initially provided with a removable seal 22, and a bottom wall 23 and contains a suitable buffer 20 for preparing the sample for analysis. The seal 22 is removed so the container 12 can be attached to the homogenizing body 16. The seal 22 may be a disposable cap, a pull-off hermetically sealed cover (not shown), and other suitable mechanisms. In one embodiment, the container 12 is sealed with a solid screw cap 24, shown in FIG. 1, which acts as a removable plug. The cap 24 has external threads 24a that mate with the internal threads 12a of the container 12.

The container 12 is sized so as to adequately and appropriately homogenize the sample for analysis. Typically, in the container 12, the collected sample and buffer generally have a volume (cc)-to-volume (cc) ratio of sample-to-buffer ranging from about 0.001:1 to about 5:1 or greater, depending upon the fluidity of the sample. Preferably, the volume (cc)-to-volume (cc) ratio of sample-to-buffer ranges from about 0.05:1 to about 1:1, and most preferably from about 0.05:1 to about 0.1:1.

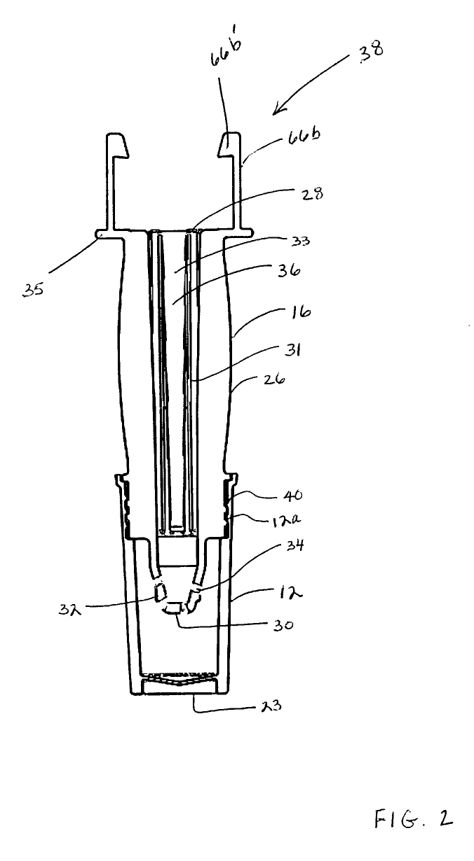

FIG. 2 is a cross-sectional view, showing the homogenizing body 16 attached to the container 12 to form a homogenizing composite 38. As shown in FIGS. 2-4, the homogenizing body 16 comprises a main body portion 26 with an open end 28, an opposed end 30 having a homogenizing face 32 with at least one pore 34 therein, and a bore 36 therebetween. In one embodiment, near the open end 28, the body portion 26 has one or more flanges or rings 31 extending from an internal surface 33 into the bore 36 to support the sampling assembly 14 when the sampling assembly is inserted into the bore 36. In another embodiment, as shown in FIG. 1, to make the homogenizing body 16 stronger and easier to grasp and maneuver, the homogenizing body 16 has molded on the external surface of the body portion 26 one or more longitudinal ridges 35 or other structure such as a lip, flange, or furrow, as examples.

By way of example, the attachment of the homogenizing body 16 to the container 12 may be by attachment structures 40 disposed on the homogenizing body 16, between the two ends 28,30 which structures correspond to complementary structures 12a near or at the open end of container 12. Such attachment structures 40,12a may take the form of ridges or threading (on the body 16 and internal on the container 12) for screwing the two components together, lips and channels, mating extensions, or any other well known and suitable form of attachment.

The homogenizing face 32 has a shape and dimension (e.g., conical) allowing it to extend at least partially into the container 12 and preferably to a point near the bottom wall 23 of the container 12 so the sample may be extruded through the pores 34 directly into the buffer 20. When the homogenizing body 16 is attached to the container 12, the homogenizing face 32 extends into the buffer 20.

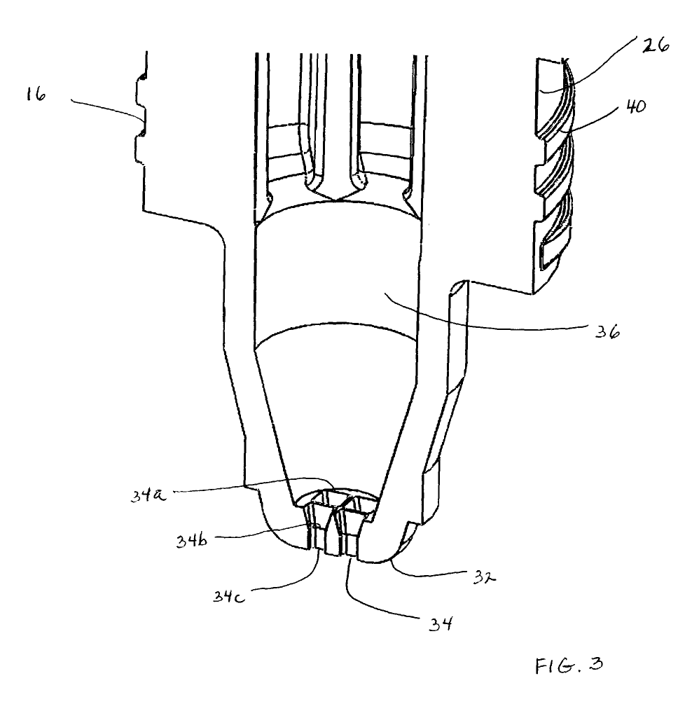

The at least one pore 34 of the homogenizing face 32 may have a variety of cross-sectional shapes chosen from round, elliptical, oblong, or amorphous, to a shape having n sides where n is an integer of 3 or greater. In one embodiment, shown in FIG. 3, the at least one pore 34 may be substantially conical or frustoconical in cross-section. The pores 34 are, in essence, open channels having a first opening 34a in communication with the bore 36, a throat 34b narrowing the passageway to the outer opening 34c.

The outer opening 34c may be of any suitable size. Typically, outer opening 34c ranges from about 0.1 to about 10 millimeters at its widest point. Preferably, the pores have an opening 34c ranging from about 0.1 to about 1 millimeter, and most preferably from about 0.25 to about 0.5 millimeters.

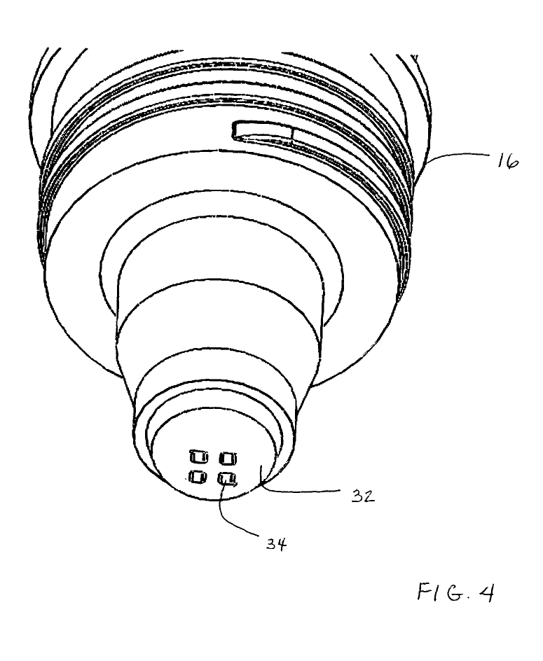

Although the homogenizing face 32 generally has at least one pore, the number of pores may range from 1 to 20, and preferably from 4 to 8. In one embodiment, the at least one pore 34 may be configured to form a sieve, screen or strainer on the homogenizing extruding face 32, as shown in FIG. 4 (four (4) pores shown).

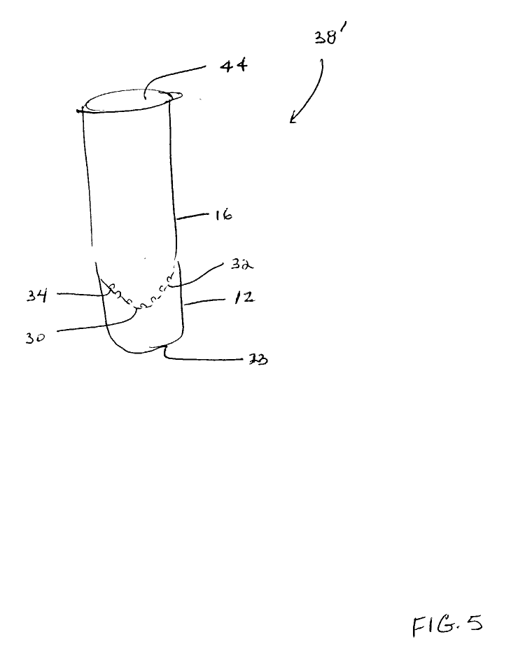

Typically, the homogenizing body 16 and the container 12 are releasably attachable; i.e., they can be attached and separated as desired. However, in an alternative embodiment, shown in FIG. 5, the homogenizing composite 38′ is a unitary piece. This composite 38′ has a removable seal 44 at the open end 28 of the body 16 and holds a suitable buffer in the container 12.

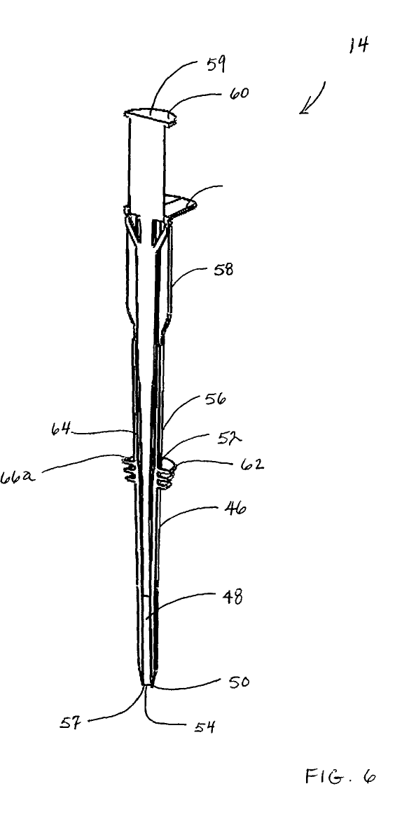

FIG. 6 shows a cross-sectional view of a sampling assembly 14 having a housing 46, defining a sampling recess 48, a first end 50, and a second end 52. There is at least one opening 54 through the housing 46 to the recess 48 generally disposed at the first end 50, and a plunger 56, or other suitable structure, slidable from the second end 52 and through the recess 48 for discharging the sample from the sampling assembly 14 into the homogenizing body 16.

The plunger 56 generally comprises an elongated member 58 having an outer distal end 59 and a cross-sectional shape and size that correspond substantially with that of the recess 48 of housing 46 such that the plunger is slidable within the recess 48. In the embodiment shown in FIG. 6, the plunger 56 may have a flange 60 or other structure at the outer distal end 59 that the user depresses to slide the plunger 56 into the recess 48. The housing 46 typically has structures such as a ridge 62 for controlling the length of the sampling assembly 14 that may be inserted into the homogenizing body 16. Effectively, when the housing 46 is inserted into the homogenizing body 16, the housing 46 slides into the bore 36 until the ridge 62 abuts against the open end 28 of the homogenizing body 16. The plunger 56 has at least one vertical ridge 64 that helps stabilize the position of the plunger 56 as it slides into the housing 46.

Generally, in the sampling assembly 14, the opening 54 for sample collection is disposed at the first end 50, as shown in FIG. 6. In such embodiment, the opening 54 is capable of serving as both an inlet for collecting the sample and an outlet for discharging the sample into the homogenizing body 16. For sample collection, the opening 54 generally has an edge 57 sufficiently sharp for slicing or otherwise extracting samples of the tissue or fluid to be analyzed so the sample readily enters the recess 48.

Typically, the user attaches the sampling assembly 14 to the homogenizing body 16 after a sample has been collected in the sampling assembly 14. The sampling assembly 14 is attached to the homogenizing body 16 after the homogenizing body 16 has previously been secured to the container 12 to form the homogenizing composite 38.

The sampling assembly 14 may be secured to the homogenizing body 16 by an attachment structure 66a on the housing 46 of the sampling assembly 14 that interconnects with a corresponding structure 66b′ on the homogenizing body 16. For example, as shown in FIGS. 1 and 2, the homogenizing body 16 has a flange 66b with an inwardly projecting hook 66b′ that interconnects with a ring 66a extending outwardly from the housing 46 of the sampling assembly 14. This configuration allows the homogenizing body 16 and the sampling assembly 14 to be snapped together. Any suitable means for attachment may, however, be used, including a screwing mechanism and other devices.

The sampling assembly 14 has a configuration and size that substantially correspond with that of the homogenizing body 16, and is slightly narrower than the homogenizing body 16, so that at least a portion of the sampling assembly 14 may be inserted into the homogenizing body 16. When attached to the homogenizing body 16, the first end 50 of the sampling assembly 14 fits into the open end 28 of the homogenizing body 16 and extends downwardly towards the homogenizing face 32. The plunger 56 is made to slide towards the homogenizing face 32, so that sample in the recess 48 may be extruded through the pores 34 of the homogenizing face 32.

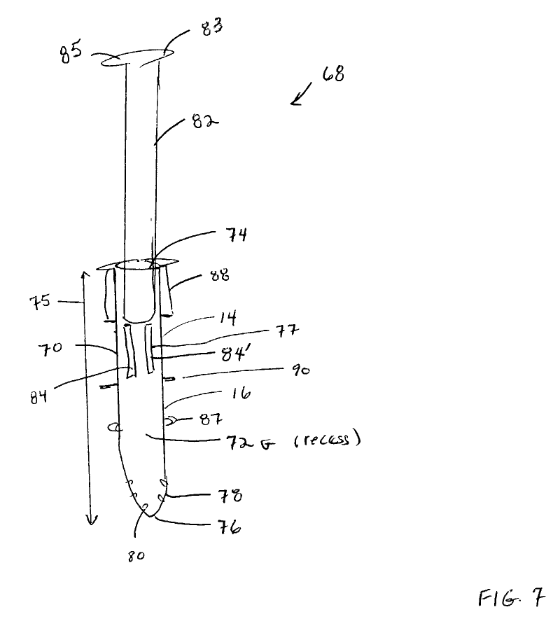

In yet another aspect of the invention, shown in FIG. 7, the sampling assembly 14 and the homogenizing body 16 may be fabricated as a single unit, called a composite sample comminuter 68. The composite sample comminuter 68 includes a housing 70, defining a sampling recess 72 therein, an open end 74, and an opposed end 76 having an extruding face 78 with at least one pore 80 therethrough. The comminuter 68 also has a plunger 82 slidable through the open end 74 and through the sampling recess 72 towards the homogenizing face 78, and at least one opening 84 for sample collection disposed in the housing 70 along a longitudinal axis 75. The opening 84 has a razor sharp edge 77 that allows the comminuter 68 to be scraped against the surface of a tissue mass for collection of a sample. The plunger 82 is generally configured as described above—i.e., with a distal end 83 having a flange 85 to prevent the plunger 82 from falling into the composite sample comminuter 68.

The composite sample comminuter 68 is attached to the container 12 by, e.g., interlocking structures 87 that mate with corresponding structures on the container 12, described above. When so attached, the extruding face 78 extends at least partially into the container 12.

The composite sample comminuter 68 allows the user to collect a sample in the sampling recess 72 by scraping the housing 70 along a surface of the material to be analyzed. The opening 84 may be in a variety of shapes, including, e.g., slits. In one embodiment, shown in FIG. 7, the housing 70 has more than one opening 84,84′. The composite sample comminuter 68 of FIG. 7 includes a sleeve 88 comprising a tube with two open ends and having a width slightly largely than that of the composite sample comminuter 68. The sleeve 88 slides over the housing 70 to close the opening 84, preventing the sample from being discharged through the opening 84 as it is being extruded into the container 12. On the exterior of the housing 70 there is a rib 90 or other structure that allows the sleeve 88 to slide over the opening 84 and lock in place. Other structures that sufficiently seal the opening 84 may be employed, such as a lid or a flap.

In another embodiment, the device 10 includes a first reagent 94 for release into the container 12 in an amount sufficient to facilitate sample preparation. The first reagent 94 may be chosen from an enzyme, a chemical, a biological agent, an antibody, a marker, a polymer, and any other suitable compound, as examples.

In one embodiment, the first reagent 94 is proteinase-K for removing normal prion protein from a sample. The enzyme is used in an amount sufficient to substantially digest all PrP^{c} present in the sample. Typically, this amount is at least 20 units of enzyme activity per milligram of protein in the sample, wet weight basis. Preferably, the amount of enzyme ranges from about 20 to about 80 units of enzyme activity and, most preferably, from about 30 to about 50 units of enzyme activity per milligram of protein in the sample, wet weight basis.

The first reagent 94 may be in the form of a capsule, a pellet, a gel, a powder, a lyophilizate, an encapsulate, and a liquid, as examples. For example, the first reagent 94 may be encapsulated and contained within the buffer 20, as shown in FIG. 1, for release into the buffer 20 under predetermined conditions, such as when the buffer is heated to a specific temperature for a predetermined time. In an alternative embodiment, the first reagent 94 is disposed on at least one surface that contacts the sample, chosen from the homogenizing face 32,78, the at least one pore 34,80, the container 12, and the sampling assembly 14. In one embodiment, the first reagent is dried onto a portion of the homogenizing face. In another embodiment, the first reagent 94 may be a tablet that is dispensed into the buffer 20 after the seal 22 is removed from container 12.

In yet another embodiment, the sample preparation device 10 may further include a second reagent 96. The second reagent 96 may be chosen from an enzyme inhibitor, a chemical, a biological agent, an antibody, a marker, a polymer, and any other suitable compound. In one embodiment, the second reagent 96 is proteinase-K inhibitor. In a particular embodiment of the invention, the first reagent 96 is proteinase-K and the second reagent is proteinase-K inhibitor.

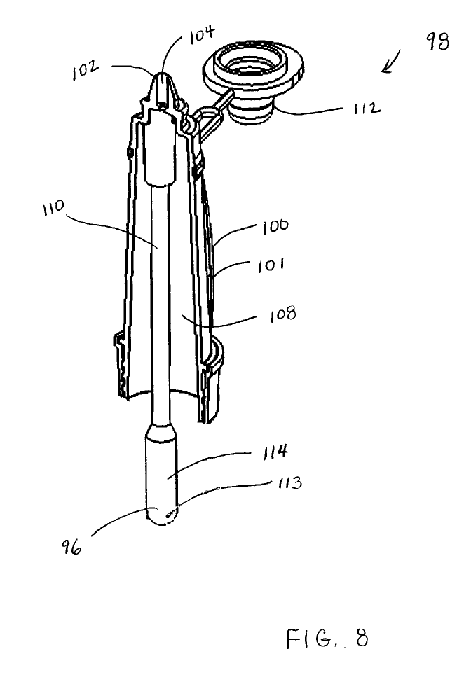

The sample preparation device 10 may further include a delivery apparatus 98 for releasing the second reagent 96 into the buffer 20 and dispensing an aliquot of sample for analysis. Shown in FIG. 8, a delivery apparatus 98 includes a dropper top dispensing component 100, an elongated dispensing member 110 having a tip 113 and being attached to the dispensing component 100, and a second reagent 96 disposed on the tip 113. In one embodiment, the tip 113 includes a tip cover 114 comprising an absorbent material such as gauze, foam, or a sponge, as examples, and having the second reagent 96 disposed thereon. The dispensing component 100 has an apparatus housing 101, a top end 102, a pore 104 at the top end 102, an opposed open end 106, and a recess 108 therebetween. The elongated dispensing member 110 is attached inside the apparatus housing 101 near the top end 102 and extends downwardly through the open end 106 of the apparatus housing 101. In one embodiment, the dispensing component includes a cap 112 for covering the pore 104 to protect it from contamination.

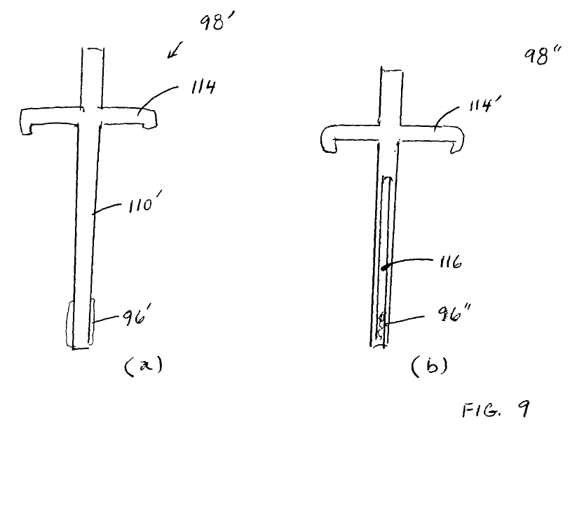

In an alternative embodiment, shown in FIGS. 9a and b, the delivery apparatus 98′ may provide solely for the release of the second reagent 96′ into the buffer 20. As shown in FIG. 9a, the delivery apparatus 98′ includes a base 114, an elongated dispensing member 110′ extending downwardly from the base 114, and a second reagent 96′ disposed on the dispensing member 110′. In one embodiment, shown in FIG. 9b, the delivery apparatus 98″ includes abase 114″, a dispensing member 110″ having a bore 116 therein, and a second reagent 96″ disposed within the bore 116 for ready release into the buffer 20.

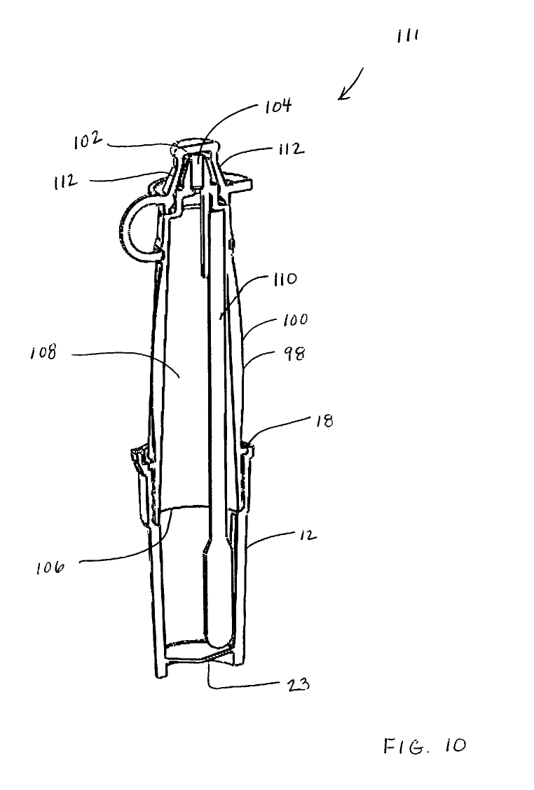

The delivery apparatus 98 is attached to the top end 18 of the container 12 to create a closed dispenser 111 as shown in FIG. 10; it is also attachable to the open end 28 of the homogenizing composite 38 (shown in FIG. 2) and 38′ (a unitary piece shown in FIG. 5). When so attached, the elongated dispensing member 110 extends at least partially into the container 12 and preferably into the buffer 20, so that the second reagent 96 is released into the buffer under defined conditions. Attachment may be by means of structures that provide for screwing, snapping, or otherwise securing the delivery apparatus 98 to the container 12. The dispenser 111 may be inverted, and the cap 112 removed to dispense sample through the pore 104 into a procedure for direct analysis.

The various components of the device 10—i.e., the sampling assembly 14, the homogenizing body 16, the container 12, and the delivery apparatus 98—are typically made by injection molding of a rigid material that is chemically inert and stable to deformation by heat. Examples of suitable materials include polyethylene, polypropylene, polycarbonate, polystyrene, and polymetacrylate.

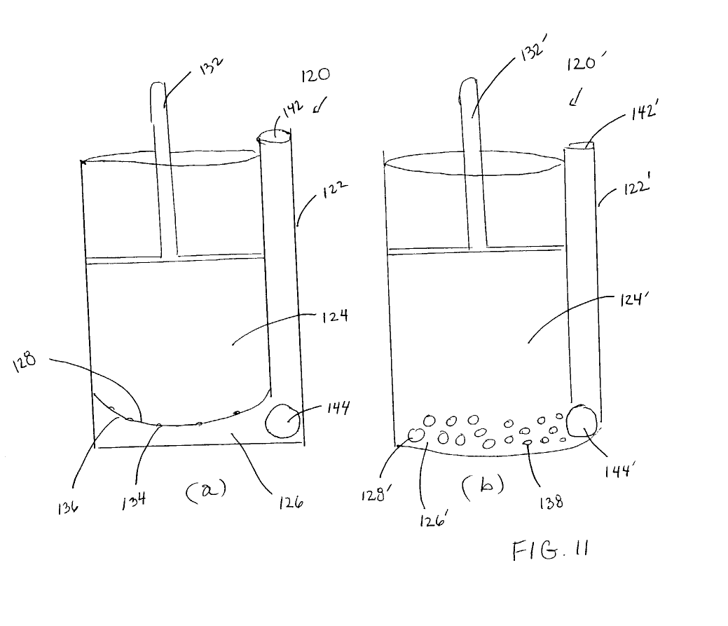

In another aspect of the invention, shown in FIGS. 11a and 11b, a device 120,120′ is provided for preparing a sample for analysis of an analyte such as pathogenic prion protein. The device 120,120′ includes a housing 122,122′ having a recess 124,124′, a sample-reaction zone 126,126′ adapted to hold a buffer, a comminuting device 128,128′, and proteinase-K. Also included are at least one opening 130,130′ in the housing 122,122′ for collecting a sample in the recess 124,124′, and a plunger 132,132′ or other means for discharging sample from the recess into the reaction zone 126. The comminuting device includes at least one of (a) at least one pore or orifice 134 on a homogenizing face 136 disposed within one of the recess 124 and the reaction zone 126, as shown in FIG. 11a; and (b) a plurality of discrete bodies 138 of an inert composition—such as glass beads or rods, as shown in FIG. 11b—disposed in the reaction zone 126′ in a quantity and having a dimension sufficient to comminute sample into particles or strands of a suitable size for analysis. The comminuted sample typically has a cross-sectional width ranging from about 0.1 to about 10 millimeters and preferably from about 0.1 to about 2 millimeters. The proteinase-K is in the form chosen from a capsule, a pellet, a gel, a powder, a lyophilizate, an encapsulate, and a liquid, as examples, that is releasable into the reaction zone 126,126′ for removing interfering protein from the sample as to yield an enzyme-treated sample. In the embodiment shown, the enzyme 144,144′ is in the reaction zone in a pellet form. In another embodiment, the device includes a dispensing pore 142,142′ or other suitable structure for dispensing the enzyme-treated sample for direct analysis of the analyte.

B. The Methods

In another aspect of the invention, a method is provided for preparing a sample for analysis for a particular analyte. The sample is a biological sample, such as brain tissue, for example. In an alternative embodiment, the sample may be food, such as hamburger meat. In one embodiment, the analyte is pathogenic prion protein.

The method includes providing a sample preparation device, substantially as described above—i.e., having a container 12 with a buffer, a homogenizing body 16, and a sampling assembly 14. The homogenizing body 12 has a body portion 26 defining a bore 36 therein, an open end 28, and an opposed end 30 having a homogenizing face 32 with at least one pore 34; the homogenizing body 16 being attached to the container 12 to form a homogenizing composite 38 wherein the at least one pore 34 extends into the container 12. The sampling assembly 14 includes a housing 46 defining a recess 48 therein, at least one opening 54 for collecting a sample in the recess, and means for discharging the sample into the buffer. The sampling assembly 14 is attached to the homogenizing body 16 so the recess 48 communicates with the open end 28 of the homogenizing body 16.

The method also includes (a) collecting a sample through the opening 54 in the sampling assembly 14; (b) securing the sample-containing sampling assembly 14 to the homogenizing composite 38 to form an assembled sample preparation device 10; (c) comminuting the collected sample by discharging the sample through the at least one pore 34 into the container 12 to form a homogenate; and (d) mixing the homogenate with the buffer in the container 12 to yield a sample ready for analysis. Depending upon the structure of the sample, the comminuted sample may be in the form of “noodles” or “strands.” Such samples have a diameter that substantially corresponds to the pore size. Therefore, in one embodiment, the comminuted sample may have a width ranging from about 0.1 to about 10 millimeters; preferably, of 2 millimeters or less; and most preferably, of 1 millimeter or less.

The buffer is selected for suitability for the particular analyte being extracted. Because the selection of the buffer is generally known to those skilled in the art, further detail is not provided herein.

In another embodiment, the method further includes adding a first reagent 94 to the buffer in an amount sufficient to facilitate sample preparation. The first reagent 94 may be a chemical, an enzyme, a biological agent, an antibody, a marker, a polymer, or other suitable compound that reacts with or binds to a constituent in the sample.

In one embodiment, the first reagent is proteinase-K which is added in an amount sufficient to substantially digest all interfering protein in the sample. Proteinase-K is typically added in an amount of at least 20 units of enzyme activity per milligram of protein in the sample, wet weight basis. Per milligram of protein in the sample, wet weight basis, the amount of enzyme preferably ranges from about 20 to about 80 units of enzyme activity and most preferably, from about 30 to about 50 units of enzyme activity.

After the first reagent 94 has been added, sufficient time and temperature are provided to allow the first reagent 94 to act upon the targeted constituents in the sample. In one embodiment, where the first reagent 94 is proteinase-K, the enzyme is allowed to act upon the sample for a time and a temperature sufficient to substantially digest the nonpathogenic prion protein. This is generally accomplished by heating the enzyme-containing homogenate in the assembled sample preparation device at a temperature and for a time sufficient for the proteinase-K to denature interfering constituents. Typically, the temperature ranges from about 30 to about 40 degrees Centigrade and the time ranges from about 30 seconds to about 30 minutes. Preferably, the enzyme-containing homogenate is heated at a temperature ranging from about 34 to about 38 degrees Centigrade and for a time ranging from about 5 to about 10 minutes.

In one embodiment, the method includes treating the sample with a second reagent 96, which may be an enzyme inhibitor, a chemical, a biological agent, an antibody, a marker, a polymer, and any other suitable compound. In one embodiment, where the first reagent is proteinase-K, the second reagent is proteinase-K inhibitor. The second reagent 96 is introduced by providing a delivery apparatus 98, substantially as described above, having an elongated member 110 with the second reagent 96 disposed on the elongated member 110. The delivery apparatus 98 is attached to the open end 18 of the container 12 to form a closed system 111, wherein the elongated member 110 extends at least partially into the container 12 and preferably into the buffer 20. Upon contacting the buffer, the second reagent 96 is released into the treated sample. Typically, when the second reagent 96 is an enzyme inhibitor, it is added in an amount sufficient to substantially inactivate the enzyme.

In another embodiment, after addition of the second reagent 96, the method optionally includes heating the homogenate at a temperature and for a time sufficient to allow the second reagent 96 to act upon the sample. When the second reagent 96 is proteinase-K inhibitor, the reaction is conducted at a temperature and for a time sufficient to remove the enzyme inhibitor and interfering constituents and to form an analysis-ready sample. Typically, the heating temperature ranges up to about 100 degrees Centigrade and preferably from about 25 to about 37 degrees Centigrade. The heating time generally ranges from about 1 minute to about 30 minutes and preferably from about 1 to about 7 minutes.

The method may further include dispensing the analysis-ready sample into at least one test container for analysis of the desired analyte. This may be done by inverting the delivery apparatus 98, removing the cap 112 so the analysis-ready sample may be dispensed through the pore 104 into a procedure for direct analysis. Such procedures may include analyses for detecting and optionally quantifying the concentration of the targeted analyte. For example, the analysis-ready sample may be dispensed into a device employing a procedure selected from the group consisting of immunoassay, immunochromatography, radio immunoassay, optical immunoassay, enzyme immunoassay, and chemiluminescence. In one embodiment, the method includes dispensing the analysis-ready sample into a lateral flow device for detecting and/or quantifying pathogenic prion protein in the sample.

In an alternative embodiment, the first reagent and the second reagent may react with the sample to produce a colored or fluorescent moiety that may be detected in the container visually or with instrumentation. In this embodiment, the method includes examining the contents of the container for fluorescence or a color change that indicates the presence of the analyte. Alternatively, the homogenate may be dispensed into cuvettes or other suitable test containers for, e.g., spectrophotometric analysis.

The method of this invention is capable of preparing a sample for analysis within from about 5 to about 45 minutes from the time the sample is collected into the device. Because of its simplicity and ease-of use, the method offers a broad range of applications. For example, it may be used solely for collecting and homogenizing a food or biological sample for subsequent analysis of a host of analytes, such as pathogenic prion protein, pathogens such as Escherichia coli and Salmonella species, metals, environmental toxins, agricultural additives such as pesticides and herbicides, genetic markers in grains, and food.

The method may also be used not only for collecting and homogenizing a sample but also treating the homogenized sample with a non-enzymatic compound. For example, to prepare a ground meat sample for analysis of E. Coli, the first reagent 94 may be a first binding antibody which binds to an epitope on the bacteria. A second reagent, optionally added, may be a second antibody conjugate attached to a visible bead or other response-producing moiety such as an enzyme, a fluoro-probe, a radioisotope, or a chemiluminescent group, as examples.

Another aspect of the invention is a method suitable for use by meat processors and others for testing a biological sample, such as brain tissue, for prion disease. Typically, the mass of sample is collected by a veterinarian or other individual and shipped with an identification code to the laboratory where the testing will take place. The sample identification code is recorded on the sampling apparatus, the homogenizing body, and the container.

The method includes (a) providing a device 10 labeled with the identification code of the animal whose biological sample—such as brain tissue—is being tested. The device is substantially as described above, in that it includes a container 12 with a buffer 20, the container being initially sealed, as with cap 24, and a homogenizing body 16 having an open end 28, an opposed end 30 having at least one pore 34, and a bore 36 therebetween. The homogenizing body 16 is attached to the container 12 so the at least one pore 34 extends into the container 12. Also included in the device is a sampling assembly 14 having a housing 46 defining a recess 48, at least one opening 54 to the recess, and a plunger 56 slidable in the recess. The sampling assembly 14 is attachable to the homogenizing body 16 so the at least one opening 54 is in communication with the bore 36 of the homogenizing body.

The device further comprises proteinase-K disposed on the homogenizing body in an amount sufficient to substantially remove nonpathogenic prion from the sample, and proteinase-K inhibitor releasable into the container in an amount sufficient to inactivate the proteinase-K. The proteinase-K inhibitor is disposed on the tip 113 of the dispensing apparatus 98.

The cap 24 is removed from the container 12, and the homogenizing body 16 is attached to the container. The next steps of the method include (b) dissolving the proteinase-K in the buffer in the container attached to the homogenizing body; (c) collecting a sample with the sampling assembly; (d) securing the sample-containing sampling assembly to the homogenizing body to form an assembled sample preparation device; and (e) comminuting the sample by extruding the sample through the at least one pore into the enzyme-containing buffer upon pushing the plunger into the sampling assembly. As the comminuted sample falls into the buffer, it forms an enzyme-containing homogenate. The container is then gently agitated to release into the enzyme-containing homogenate any comminuted sample clinging to the homogenizing face. The enzyme-containing homogenate is next subjected to at least one heating step. In a first heating step (f), the homogenate in the assembled device is heated in a heat block at a temperature and for a time sufficient for the proteinase-K to remove interfering constituents and yield an enzyme-treated sample. The homogenizing body and sampling assembly are then removed, and the dispensing apparatus 98, described above, is attached in their place to form the dispenser 111. The dispenser 111 is gently shaken to release the proteinase-K inhibitor from tip 113 into the enzyme-treated sample. In an optional second heating step (h), the inhibitor-containing sample is heated at a temperature and for a time sufficient for the proteinase-K inhibitor to remove the enzyme and yield an analysis-ready sample. The sample is then cooled to room temperature for about 5 minutes. The sample is then dispensed into a device utilizing immunochromatography, such as a lateral flow device.

The first heating step is typically conducted at a temperature ranging from about 30 to about 40 degrees Centigrade and for a time ranging from about 30 seconds to about 30 minutes. Preferably, the enzyme-containing homogenate is heated at a temperature ranging from about 34 to about 38 degrees Centigrade and for a time ranging from about 5 to about 10 minutes. In addition, the second heating step is generally conducted at a temperature of up to about 100 degrees Centigrade and for a time ranging from about 1 minute to about 30 minutes. Preferably, the second heating step is conducted at a temperature ranging from about 25 to about 37 degrees Centigrade and for a time ranging from about 1 to about 7 minutes.

While the specific embodiments have been illustrated and described, numerous modifications come to mind without significantly departing from the spirit and scope of the invention. All such modification are intended to be within the scope of the accompanying claims.

We claim:

1. A method of preparing a sample for analysis for a particular analyte comprising:
(a) providing a sample preparation device having
a container with a buffer;
a homogenizing body having a body defining a bore therein, an open end, and an opposing end having at least one pore; the homogenizing body being attachable to the container to form a homogenizing composite wherein the at least one pore extends into the container; and
to a sampling assembly having a housing defining a recess therein, at least one opening for collecting a sample, and means for discharging the sample into the buffer, the sampling assembly being attachable to the homogenizing body so as to interface with the open end of the homogenizing body;
(b) collecting a sample through at least one opening of the at least one opening in the sampling assembly; and
(c) comminuting the sample by extruding the sample through the at least one pore of the homogenizing body into the buffer to form a homogenate ready for analysis.

2. The method of claim 1 including adding a first reagent to the homogenate in an amount sufficient to facilitate sample preparation.

3. The method of claim 2 wherein the first reagent is an enzyme.

4. The method of claim 3 further including allowing the enzyme to act upon a targeted substrate to form an enzyme-treated sample for analysis of an analyte.

5. The method of claim 2 wherein the first reagent is proteinase-K added in an amount sufficient to substantially digest nonpathogenic prion protein in the sample and yield an enzyme-treated sample.

6. The method of claim 1 wherein the proteinase K is added at a amount of at least 20 units of enzyme activity per milligram of protein in the sample, wet weight basis.

7. The method of claim 5 further including heating the homogenate at a temperature and for a time sufficient for the proteinase-K to denature nonpathogenic prion protein and yield an enzyme-treated sample.

8. The method of claim 7 wherein the heating temperature ranges from about 30 to about 40 degrees Centigrade and the time ranges from about 30 seconds to about is 30 minutes.

9. The method of claim 7 wherein the heating temperature ranges from about 34 to about 38 degrees Centigrade and the time ranges from about 5 to about 10 minutes.

10. The method of claim 2 further including adding a second reagent to the homogenate.

11. The method of claim 5 further including adding proteinase-K inhibitor to the enzyme-treated sample in an amount sufficient to inactivate the proteinase-K.

12. The method of claim 11 wherein the step of adding the proteinase-K inhibitor includes contacting the enzyme-treated sample with a dispensing member having the inhibitor disposed thereon.

13. The method of claim 11 further including heating the enzyme-treated sample containing the enzyme inhibitor to a temperature of up to about 100 degrees Centigrade and for a time ranging from about 1 minute to about 30 minutes.

14. The method of claim 10 further including heating the homogenate containing the second reagent at a temperature and for a time sufficient to allow the second reagent to act upon the sample.

15. The method of claim 2 wherein the sample is a biological sample.

16. The method of claim 5 wherein the sample is food.

17. The method of claim 1 further including dispensing the sample into at least one test container for analysis of the analyte.

18. The method of claim 1 further including dispensing the sample into a system employing a procedure selected from the group consisting of immunoassay, immunochromatography, radio immunoassay, optical immunoassay, enzyme immunoassay, and chemiluminescence.

19. The method of claim 2 further including detecting the analyte by examining the homogenate in the container for a moiety consisting of a compound from the group comprising a colored moiety and a fluorescent moiety.

20. The method of claim 5 further including dispensing the sample into a system employing a procedure selected from the group consisting of immunoassay, immunochromatography, radio immunoassay, optical immunoassay, enzyme immunoassay, and chemiluminescence.

21. The method of claim 1 wherein the analyte is pathogenic prion protein.

22. The method of claim 5 further including dispensing the sample into a lateral flow device that detects the analyte.

23. The method of claim 22 wherein the sample is prepared for analysis within from about 5 to about 45 minutes from the time the sample is collected into the device.

24. The method of claim 1 wherein the comminuting step yields a sample having a size ranging from about 0.1 to about 10 millimeters in width.

25. A method for preparing a biological sample from an animal for prion analysis, comprising:
(a) providing a device labeled with an identification code of the animal whose tissue is being tested; the device including
a container containing a buffer;
a homogenizing body having an open end, an opposed end having at least one pore, and a bore therebetween and being attachable to the reaction vessel so the at least one pore extends into the reaction vessel;
a sampling assembly having a housing defining a recess, at least one opening to the recess, and a plunger slidable in the recess, and being attachable to the homogenizing body so the at least one opening is in communication with the bore of the homogenizing body;
proteinase-K disposed on the homogenizing body in an amount sufficient to substantially remove nonpathogenic prion from the sample; and
proteinase-K inhibitor releaseable into the container;
(b) dissolving the proteinase-K in the buffer in the container attached to the homogenizing body;
(c) collecting a sample with the sampling assembly;
(d) securing the sampling assembly to the homogenizing body;
(e) comminuting the sample by moving the sample through the at least one pore into the buffer to form an enzyme-containing homogenate;
(f) in a first heating step, heating the enzyme-containing homogenate at a temperature and for a time sufficient for the proteinase-K to remove interfering constituents and yield an enzyme-treated sample;
(g) releasing the proteinase-K inhibitor into the enzyme-treated sample upon attaching the dispensing apparatus to the container;
(h) in a second heating step, heating the inhibitor-containing sample at a temperature and for a time sufficient for the proteinase-K inhibitor to remove the enzyme and yield an analysis-ready sample; and,
(i) dispensing the analysis-ready sample into a lateral flow analytical device.

26. The method of claim 25 wherein the first heating step is conducted at a temperature ranging from about 30 to about 40 degrees Centigrade and for a time ranging from about 30 seconds to about 30 minutes.

27. The method of claim 25 wherein the second heating step is conducted at a temperature of up to about 100 degrees Centigrade and for a time ranging from about 1 to 30 minutes.

28. The method of claim 25 wherein the dispensing step comprises dispensing the analysis-ready sample into a lateral flow analytical device utilizing immunochromatography.
